(19) United States
(12) Patent Application Publication
(10) Pub. No.: US 2015/0238733 A1
(43) Pub. Date: Aug. 27, 2015

(54) CATHETER DEVICES AND RELATED METHODS

(71) Applicant: Mohamad Yasin bin Abdulla, Penang (MY)

(72) Inventor: Mohamad Yasin bin Abdulla, Penang (MY)

(21) Appl. No.: 14/610,951

(22) Filed: Jan. 30, 2015

Related U.S. Application Data
(60) Provisional application No. 61/944,181, filed on Feb. 25, 2014.

(51) Int. Cl. A61M 25/06; A61M 25/00

(52) U.S. Cl. CPC A61M 25/0637; A61M 25/0014

(57) ABSTRACT

Catheter devices and their components are disclosed. The catheter devices described each includes a catheter hub, a needle guard housing, and a needle hub. The needle guard housing is removably secured to the exterior of the catheter hub with a positive engagement mechanism. In some examples, the positive engagement mechanism can include detents and leaf springs.

### CROSS REFERENCE TO RELATED APPLICATION

The application claims priority to U.S. Provisional Application No. 61/944,181, which was filed on Feb. 25, 2014, the entirety of which is hereby expressly incorporated herein by reference.

### FIELD OF ART

The present invention relates generally to needle assemblies, catheter devices and related methods. In particular, the disclosed catheter devices include an ergonomic needle grip for a medical device. More particularly, the needle grip is especially adapted for use with intravenous catheters.

### BACKGROUND

In order to properly place medical devices such as intravenous (“IV”) catheters into a patient, the catheter tube is typically mounted over an introducer needle having a sharp distal tip which extends past the distal tip of the catheter tube. The catheter tube and introducer needle are inserted at a shallow angle through the patient's skin and into a vein. Typically, the clinician confirms that there is blood in a flashback chamber incorporated into the needle hub assembly in order to verify proper placement of the catheter in the vein. Once the catheter is properly inserted, the clinician withdraws the needle, leaving the catheter in place for use in accordance with standard medical techniques.

It is common for clinicians to use varying techniques for inserting a catheter tube into a patient. These varying techniques are due in part to comfort of the clinician and in part to the fact that different types of catheters are available on the market. Two common types of commercially available catheters are straight and ported catheters. A ported catheter typically includes a radially extending side port integral with the catheter hub. (See for example the catheter disclosed in U.S. Pat. No. 5,098,405). Ported catheters are commonly used in Europe. With a ported catheter, the clinician typically grasps the assembly by placing the thumb on a grip plate, one finger on the side port of the catheter adapter and another finger on a front edge of a catheter hub wing. Alternatively, the clinician may grasp the assembly by placing the thumb on the grip plate and the forefinger or middle finger on the side port of the catheter adapter. A straight catheter, on the other hand, does not include a side port for connection to a fluid handling device. Thus, the fluid handling device is instead connected to the proximal end of the catheter hub. (See for example the catheters disclosed in U.S. Pat. Nos. 4,193,400 and 5,685,855). Such straight catheters are typically used in the United States. With a straight catheter, the clinician typically grasps the assembly by placing the thumb and forefinger or middle finger of one hand on either side of the needle hub.

Once a clinician learns a particular technique to insert a catheter into a patient, that clinician will likely continue to use that technique to insert catheters into future patients.

### SUMMARY

The various embodiments of the present safety needle assembly and catheter device and methods have several features, no single one of which is solely responsible for their desirable attributes. Without limiting the scope of the present embodiments as expressed by the claims that follow, their more prominent features now will be discussed briefly. After considering this discussion, and particularly after reading the section entitled “Detailed Description,” one will understand how the features of the present embodiments provide the advantages described herein.

In a first example, a catheter device is provided comprising: a catheter hub comprising hub body with a first detent element on an exterior surface; a needle guard housing in contact with the catheter hub and comprising a second detent element on an interior surface; a needle hub in contact with the needle guard housing and comprising a needle comprising a shaft and a distal tip; and wherein the first detent element is engaged to the second detent element to prevent accidental separation between the catheter hub and needle guard housing.

The device wherein the first detent element can comprise a groove having two sidewalls having a v-shape cross-sectional end view.

The device wherein the first detent element can comprise a groove having two sidewalls and a bottom wall.

The device wherein both the first and second detent elements can take the form of projections.

The device wherein one of the detent elements can comprise external threads.

The device can further comprise a tether connecting the needle hub and the needle guard housing.

The device can further comprise a change in profile formed proximally of the distal tip of the needle.

The device can further comprise a perimeter defining an opening on an interior of the needle guard housing.

The device wherein the change in profile can engage the perimeter to separate the needle guard housing from the catheter hub.

Another aspect of the present disclosure is a method of manufacturing a catheter device. In an example, the method of manufacturing can comprise: forming a catheter hub with a catheter tube; said catheter hub comprising an exterior surface with exterior threads; forming a needle guard housing with a wall surface having an exterior surface and an interior surface defining an interior cavity; placing the needle guard housing over a proximal end of the catheter hub such that the proximal end of the catheter projects into the interior cavity of the needle guard housing; forming a needle hub with a needle having a needle tip and placing the needle hub into engagement with the needle guard housing such that the needle projects through the needle guard housing, the catheter hub, and the catheter tube and the needle guard housing is located inside a shroud formed with the needle hub; and removably securing the needle guard housing with the catheter hub by engaging a first detent element on the exterior surface of the catheter hub with a second detent element in the interior cavity of the needle guard housing.

The method wherein the first detent element can be a groove and the second detent element can be a projection.

The method wherein the first detent element can be the exterior threads on the catheter hub.

The method can further comprise a tether attached to the needle guard housing and the needle hub.

The method wherein the needle can comprise a change in profile formed proximally of the needle tip for engaging a perimeter defining an opening formed with the needle guard housing.

The method wherein the second detent element can comprise two leaf springs formed on two sidewalls of the needle guard housing.

Another aspect of the present needle device embodiment is a catheter device comprising: a catheter hub comprising a first detent element; a needle guard housing in contact with the catheter hub and comprising a second detent element; a needle hub in contact with the needle guard housing and comprising a needle, the needle comprising a shaft, and a distal tip; wherein engagement between the first detent element and second detent element increases the force required to separate the catheter hub from the needle guard housing in order to prevent accidental separation of the catheter hub and needle guard housing.

Another aspect of the present disclosure is a method for rendering a catheter device safe. In an example, the method can comprise: applying a proximally directed force to a needle hub to move the needle hub out of contact with a needle guard housing; withdrawing a needle from the catheter tube and catheter hub and into the needle guard housing; applying sufficient force to overcome the engagement of a first detent element on the catheter hub and a second detent element on the needle guard housing; moving the needle guard housing, needle and needle hub away from the catheter hub.

The method wherein the first detent element can comprise a groove and the second detent element can comprise a projection.

The method wherein both the first detent element and the second detent element can comprise projections.

Yet another feature of the present disclosure is a catheter device comprising: a catheter hub comprising a first detent element; a needle guard housing in contact with the catheter hub and comprising a second detent element; a needle hub in contact with the needle guard housing and comprising a needle, the needle comprising a shaft, and a distal tip; wherein the catheter device comprises a first position in which the needle shaft passes through the needle guard housing, and the distal tip of the needle is located distally of the needle guard housing, and the needle guard housing is in contact with the catheter hub and the needle hub, to a second position wherein the distal tip of the needle is located inside the needle guard housing, and the needle guard housing is not in contact with the catheter hub or the needle hub.

The device wherein the first detent element can comprise a groove having two sidewalls in a v-shape.

The device wherein the first detent element can comprise a groove having two sidewalls and a bottom wall.

The device wherein both the first and second detent elements can take the form of projections.

The device wherein one of the detent elements can comprise exterior threads on the catheter hub.

The device can further comprise an engagement arrangement.

The device wherein the engagement arrangement can comprise a first element and a second element.

The device wherein the first element can comprise a change in profile on the needle and the second element can comprise an aperture on the needle guard housing.

A still yet further aspect of the present disclosure is a catheter device comprising: a catheter hub comprising a first detent element; a needle guard housing in contact with the catheter hub and comprising a second detent element; a needle hub in contact with the needle guard housing and comprising a needle, the needle comprising a shaft and a distal tip; wherein engagement between the first detent element and second detent element prevents accidental separation of the catheter hub and needle guard housing; and wherein the first detent element comprises a groove and the second detent element comprises a projection, the first detent element and the second detent element are both projections, or the first detent element is a projection and the second detent element is a groove.

The device wherein the groove can have two sidewalls in a v-shape.

The device wherein the groove can have two sidewalls and a bottom wall.

The device wherein the needle guard can comprise multiple sub-components that are assembled together.

The device can further comprise a tether that unfolds and stretches out between the needle guard housing and the needle hub.

The device wherein the needle can comprise a change in profile and the needle guard housing can be provided with an engaging means to engage the change in profile.

The device wherein the engaging means can comprise a perimeter defining an opening.

### BRIEF DESCRIPTION OF THE DRAWINGS

These and other features and advantages of the present device, system, and method will become appreciated as the same becomes better understood with reference to the specification, claims and appended drawings wherein:

FIG. 1 shows a perspective view of a catheter device in a ready or initial position.

FIG. 2 shows a perspective view of the catheter device in an activated or protected configuration or position.

FIG. 3 shows a detail or close up view of a portion of a catheter device.

FIG. 4A shows a side schematic view of a detent element on the needle guard housing.

FIG. 4B shows a top cutaway schematic view of detent elements on the needle guard housing.

### DETAILED DESCRIPTION

The detailed description set forth below in connection with the appended drawings is intended as a description of the presently preferred embodiments of catheter devices provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

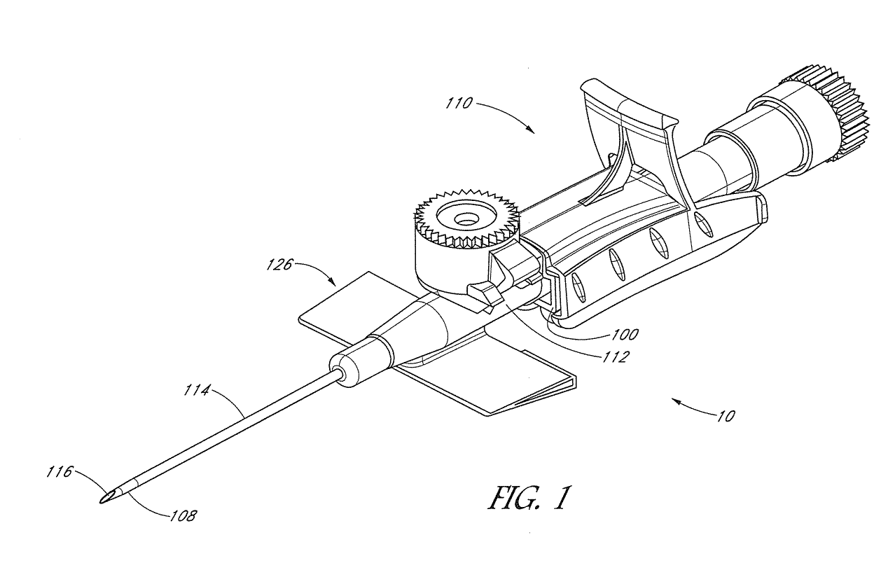

FIG. 1 shows one embodiment of a needle assembly 10 in a ready to use position prior to placement in a vein. The needle assembly may also be referred to as a catheter device or simply assembly, as an abbreviated reference. The assembly 10 comprises a needle hub 110, a needle guard housing 100, a catheter hub 112, a catheter tube 114, a pair of wings 126, and a needle 108 with a needle tip 116. Conventionally speaking, the distal end of the catheter device 10 is generally toward needle tip 116 and the proximal end is generally toward the needle hub 110, typically closer to the practitioner.

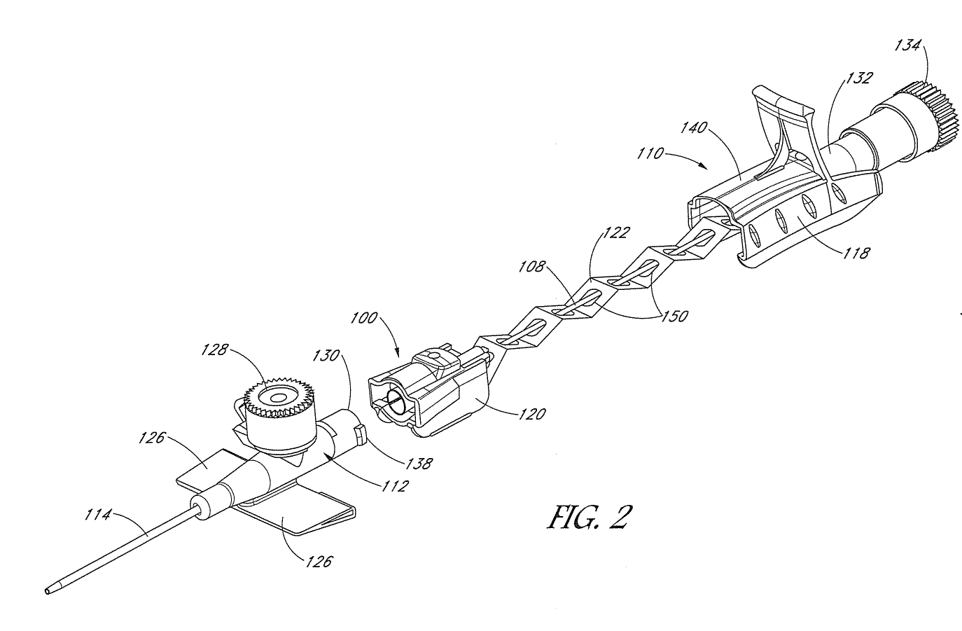

FIG. 2 shows the catheter device 10 of FIG. 1 with the needle hub 110, needle guard housing 100 and needle 108 in an activated position or withdrawn position, such as following a successful venipuncture and the catheter tube 114 is placed in a vein. The patient is omitted for clarity. In the activated position, also called a protected position, the sharp distal tip (not shown) of the needle is covered by the needle guard housing 100 to prevent inadvertent needle stick. For example, the needle tip is recessed within a distal end edge of the needle guard housing and the housing prevents accidental contact between the needle tip and the skin. The catheter device 10 also includes a grip part 118 and a tether 122, which is used to prevent the needle guard housing 100 from sliding distally off of the needle tip. The tether 122 shown is in the form of a folded or pleated string having a plurality of openings 150 for placement of the tether in-line with the needle 108. In other examples, the tether embodies a single strand or a multi-strand string placed adjacent the needle to be expanded adjacent the needle.

In other examples, the catheter device 10 is provided with an engagement arrangement with two elements. The first element of the engagement arrangement may be a change in profile, for example, a crimp or a bulge near the distal tip of the needle. The second element of the engagement arrangement may be provided with the needle guard housing as a proximal wall with a perimeter defining an opening for engaging the crimp. The opening on the proximal wall may be that of the needle guard housing if no needle guard is incorporated inside the needle guard housing. Alternatively, if a resilient needle is placed inside the needle guard housing, the proximal wall with the perimeter defining the opening may be that of the needle guard. The engagement arrangement prevents the needle guard housing from sliding distally off of the needle without a tether and allows a user to apply force to separate the needle guard housing from the catheter hub via the needle hub and needle. Where a resilient needle guard is used with the needle guard housing, the engagement arrangement with the resilient needle guard by the change in profile stops the needle guard from displacing distally off of the needle tip, which obstructs and prevents the needle guard housing from being displaced off of the needle tip. The catheter hub 112 is further shown with a catheter tube 114, wings 126, an injection port 128, a notch 130 for engaging the needle guard housing 100 in the ready to use position, and a Luer lock member or threads 138. The needle hub 110 includes a flashback chamber 132 and flow control plug 134, which can operate as a removable vent plug. Also shown with the needle hub 110 is a panel or shroud 140 with a grip part 118. The shroud 140 provides a receiving space for accommodating the needle guard housing 100 in the ready position, as shown FIG. 1. In some embodiments, the side injection port 128 is omitted from the catheter hub 112.

In use, the needle 108 of the assembled catheter device 10 shown in FIG. 1 is inserted at an angle through the patient's skin into a vein. Placement of the needle 108 with the catheter tube 114 in a vein is verified by blood flashback at the flashback chamber 132. The practitioner can then insert the needle tip further into the vein and retract the needle proximally relative to the catheter tube to permit blood flow through the annular space between the needle and the catheter tube, which is known in the industry as secondary flashback. Once successful venipuncture is confirmed, pressure is applied to the vein at the access site by pressing down on the patient's skin. This pressure occludes or at least minimizes further blood flow through the needle 108 and the catheter tube 114. The needle 108 is then withdrawn from the catheter tube 114 by pulling proximally on the needle hub 110 while leaving the catheter tube 114 in place in the patient's vein.

As the needle 108 is withdrawn from the catheter tube 114, the needle hub 110 pulls away in a proximal direction from the needle guard housing 100, which is removably secured to the catheter hub 112, as further discussed below. As this occurs, the tether 122 unfolds and straightens between the needle guard housing 100 and the needle hub 110. Further retraction of the needle hub moves the needle tip into the needle guard housing. When the needle tip 116 is withdrawn into the needle guard housing 100, which coincides with the tether 122 being stretched taut, the next proximal pull on the needle hub causes the tether to pull on the needle guard housing to disengage the needle guard housing 100 from the catheter hub 112. The needle guard housing 100 now covers the needle tip 116 to prevent accidental needle sticks and the needle guard housing separates from the catheter hub to be with the needle. The length of the tether 122 is sized such that when fully extended, the needle guard housing 100 encompasses the needle tip 116. To prevent moving the needle guard housing in a proximal position on the needle to re-expose the needle tip, a blocking wall may be incorporated with the needle guard housing. For example, a biasing arm may be positioned inside the interior of the needle guard housing and when the needle tip moves proximally of the biasing arm, the arm moves in front of the distal needle tip to prevent the distal needle tip from moving distally back out the needle guard housing.

In alternative embodiments that incorporate an engagement arrangement, including a first element such as a crimp on the needle and a second element in the needle guard housing, such as a proximal aperture with an inside diameter less than the outside diameter of the crimp, the needle guard housing will separate from the catheter hub shortly after or concurrently with the engagement between the crimp and the proximal aperture. Embodiments that incorporate an engagement arrangement preferably do not include a tether 122.

After this separation or disengagement of the needle guard housing 100 from the catheter hub 112, the clinician is then able to discard the needle 108, the needle hub 110 and the needle guard housing 100 as prescribed. It should be noted that the needle guard housing 100 shown and described in reference to FIG. 2 is only one possible embodiment of a needle guard housing that may be used with the catheter device 10. For example, a needle guard housing may be designed with multiple sub-components that are assembled together and wherein one of the sub-components comprises the second element of the engagement arrangement. Further some components may be omitted, such as the extended side walls 120 of the needle guard housing 100.

Prior to insertion of the catheter tube 114 into the patient, the needle guard housing 100 of the catheter device 10 is at least partially encased or covered by the needle hub 110, as previously mentioned and shown in FIG. 1. One benefit of such a design is to minimize the risk of early activation or withdrawal of the needle 108 from the catheter tube 114 and/or early activation or separation of the needle guard housing 100 from the catheter hub 112, where the needle tip of the needle has yet to enter into the needle guard housing. Even when the catheter device 10 is held improperly by a user by gripping some portion other than a grip 118 on the needle hub (FIG. 2), the placement of the needle guard housing 100 within the shroud 140 of the needle hub 110 minimizes the possibility that the clinician's grasp on the catheter device 10 will cause a premature separation of the needle guard housing 100 from catheter hub, thereby causing the needle 108 to withdraw from the catheter hub without first being covered by the needle guard housing. In addition, the risk of separating the needle hub 110 from the needle guard housing 100 and causing, for example, the tether 122 to tear is minimized with the noted arrangement.

With reference again to FIG. 2, the catheter hub 112 includes a pair of wings 126 and a port 128. The wings 126 provide an anchor for gripping and manipulating the catheter hub 112 during venipuncture and for securing the catheter hub 110 to the patient and the catheter tube 114 to the access site upon removal of the needle 108. The wings 126 are large enough to allow taping or other means of attachment to secure the catheter hub 112 to the patient so that once catheter tube 114 is located in the vein, its movement is minimized and it does not withdraw from the patient's body unintentionally or cause discomfort if inadvertently contacted. The port 128 provides means for introducing fluid into the catheter hub 112 and allows connection to such things as medicine or fluids to the catheter tube 114, which is conventional. In addition, the surface area provided by the wings 126 functions to reduce pressure on the patient when injecting the patient with a fluid through the injection port 128. For example, pressure applied to the port 128 is distributed by the large surface area of the two wings.

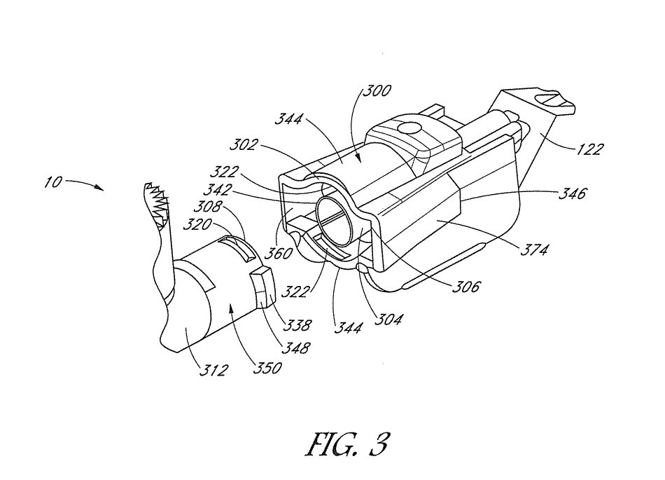

FIG. 3 shows a partial perspective view of a catheter device 10 provided in accordance with aspects of the present invention, which is similar to the catheter device of FIGS. 1 and 2. In particular, a detail view of a catheter hub 312 and a needle guard housing 300 of the alternative catheter device 10 is shown. The needle guard housing 300 comprises a wall 302, which defines an interior 304. Located near the center of the interior 304 is a cylinder shaped internal guard 342 which surrounds the needle (not shown) in a ready position and the needle tip in a protected position. The internal guard 342 extends a proximal position inside the guard housing, such as from a proximal wall of the needle guard housing 300 to an open distal end 306 of the needle guard housing 300. At the distal end 306, which is open, the needle guard housing 300 is sized and shaped to accept the proximal end 308 of the catheter hub 312, including the exterior threads 338. The body of the needle guard housing 300 is generally shaped as a rectangular parallelepiped with semi-cylindrical protrusions 344 formed at centered positions on both the top and bottom sides. The shaped body of the needle guard housing with the semi-cylindrical protrusions 344 define an interior contour that is sized and shaped to snuggly receive the catheter hub into the interior 304 of the needle guard housing 300. The body 350 of the catheter hub 312 is generally cylindrically shaped and fits into the distal opening of the needle guard housing 300, including the external threads 338 fitting into relief areas 360 to the two sides of the semi-cylindrical protrusions 344.

The generally rectangular parallelepiped configuration of the needle guard housing 300 tapers from the open distal end 306 inwardly as the wall surfaces extend in a proximal direction such that the needle guard housing 300 forms a reduced section 346, similar to a waist, generally at a location proximal of the lengthwise center. The taper shape reduced section 346 resembling a waist helps the needle guard housing 300 to enter an interior of the needle hub 110 (FIG. 2) formed by the shroud 140 on the needle hub 110. Other shapes for the needle guard housing 300 are contemplated, including a cylindrical shape, and an increased dimension parallelepiped shape that obviates the need for the semi-cylindrical protrusions. The only requirement is that the needle guard housing 300 secures the needle tip to prevent accidental needle sticks in a protective or protected position.

Included on both the proximal end 308 of the catheter hub 312 and the distal end 306 of the needle guard housing 300 are detent elements 320, 322. In an embodiment, a first detent element 320 is provided on the catheter hub 312 and embodies a groove. A second detent element 322 is provided on the needle guard housing 300 and embodies a projection sized and shaped to interact or engage the groove 312 formed on the catheter hub 312. In other embodiments, the detent elements 320, 322 are reversed. For example, the groove can be provided on the needle guard housing and the projection on the catheter hub. Rather than relying solely on friction between the exterior threads 338 and surface contacts with the two relief spaces 360 between the needle guard housing and the catheter hub, the present embodiment uses interference to removably retain the needle guard housing to the catheter hub in the ready to use position and during retraction of the needle from the catheter hub following successful venipuncture. In some examples, the first detent element 320 can be a single groove formed on the needle guard housing 300. The groove is sized and shaped to engage with the second detent element 322, which comprises a projection formed with the catheter hub. In some examples, two grooves and two projections are incorporated as first and second detent elements to facilitate securing the needle guard housing to the catheter hub. In other embodiments, greater than two first and second detent elements are contemplated, constrained only by the physical space on both the catheter hub and the needle guard housing for such corresponding pairs of detent elements.

The engagement between the first and second detent elements are provided to ensure proper activation sequence. For example, during retraction of the needle hub and needle following successful venipuncture away from the catheter hub, the needle guard housing 300 remains engaged to the catheter hub 312 while the needle and needle hub move proximally relative to the engaged components. Once the needle hub pulls the tether sufficiently taut, the constraint on the tether pulls on the needle guard housing and overcoming the engaged detent elements to separate the needle guard housing from the catheter hub. If a change in profile is used to engage a perimeter on the needle guard housing during retraction without a tether, the engagement between the change in profile and the needle guard housing will cause the two detent elements to separate and the needle guard housing to separate from the catheter hub.

In some embodiments, each groove may have two side walls in a “V” shape, when viewed in an end cross-section. In other embodiments, each groove might have two side walls and a bottom wall, similar to a “U” in cross-section. In these embodiments, the side walls might be angled or straight. In still other embodiments, the projection and groove might have rounded sides or a rounded bottom or both. In all embodiments, the detent elements 320, 322 are sized and shaped such that a minimal or incidental amount of force applied to the catheter hub 312 or needle guard housing will not result in disengagement of the detent elements 320, 322. However, when a user intends to retract the needle and needle hub following successful venipuncture or simply intends to remove the needle from the catheter tube, the catheter device 10 will move from the initial or ready position to an activated or protective position with the needle tip residing inside the needle guard housing. The needle guard housing will separate from catheter hub by either a pull by a tether or by a change in profile on the needle engaging a perimeter defining an opening on the needle guard housing and pulling on the perimeter. Thus, the projection may have an end that projects to a bottom of the groove or come short of the bottom of the groove so long as retraction of the needle and incidental contact of the needle guard housing does not separate the first detent element from the second detent element. Separation should only occur when the tether pulls on the needle guard housing in a proximal direction or when the change in profile engages a perimeter defining an opening on the needle guard housing and pulling on the perimeter.

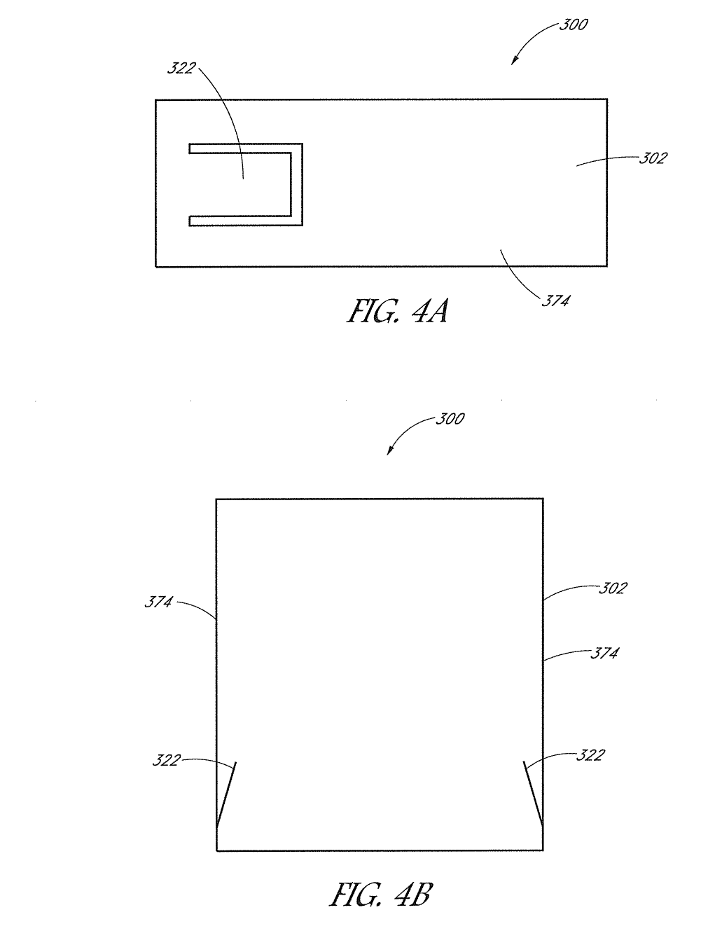

In still other embodiments, the first detent element and the second detent element may both embody as or take the form of projections. In an example, shown schematically in FIGS. 4A and 4B, the second detent element 322 comprises leaf spring projections, or simply leaf springs, extending inwardly from the wall 302 of the needle guard housing 300. In a particular embodiment, the sidewalls 374 where the reliefs 360 are provided include deflectable projections 322. In this embodiment, the second detent element 322 engages the external threads 338 on the catheter hub 312, which are also projections, to secure the needle guard housing to the catheter hub in the ready position and during retraction of the needle and needle hub from the catheter hub, such as following successful venipuncture. As shown, the leaf spring projections are formed from the two sidewalls 374 of the needle guard housing 300. For example, gaps along three edges may be provided on each of the two sidewalls 374 to form two leaf springs, as shown in FIG. 4A. In other embodiments, the leaf spring projections may be formed separately and attached to the needle guard housing through various means, including sonic welding, and mechanical methods, such as interlocking. Thus, an aspect of the present disclosure is understood to include a needle guard housing having projections for engaging an exterior of a catheter hub. In an embodiment, the projections are provided as leaf springs. In a particular example, two sidewalls 374 of the needle guard housing are each provided with three inter-connected slots to form a leaf spring. In other examples, a continuous arc is provided with two spaced apart ends for forming a leaf spring, which has a connected end that connects to the wall surfaces of the needle guard housing.

The leaf springs 322 shown in FIGS. 4A and 4B are configured to engage the external threads 338 on the exterior surface of the catheter hub 312. For example, when the catheter hub 312 projects into the distal opening 306 of the needle guard housing 300, the leaf springs 322 on the sidewalls 374 engage the external threads 338. As is shown in FIG. 3, the distal side 348 of the external threads 338 is sloped. When a user applies a proximally directed force to the needle guard housing 300, such as when a tether is taut due to the retraction of the needle hub or when a change in profile on the needle engages a perimeter defining an opening on the needle guard housing, that retraction force is transferred through the leaf spring projections 322 to the distal side of the external threads 338. Because the distal face of the threads is sloped, when retraction force is applied to the leaf spring projections 322, the leaf spring projections 322 deflect radially outward away from the central lengthwise axis of the catheter device until the ends of the leaf spring projections 322 clear the outside radial edges of the external threads 338, thereby releasing the needle guard housing 300 from the catheter hub. Depending on the length and/or placement of the leaf springs 322 on the needle guard housing, the leaf springs 322 can engage or contact the side edges of the threads 338 and the angular arrangement of the leaf springs trapping the external threads 338 to create an interference. As disclosed, the interference can change by deflecting the leaf springs 322, such as by holding the catheter hub and pulling on the needle guard housing with a tether or pulling on the needle guard housing through an engagement between a change in profile on the needle and a perimeter defining an opening formed with the needle guard housing to cause the leaf springs to deflect as they move proximally past the external threads 338. Note that when a perimeter defining an opening is described in connection with a needle guard housing, it is understood to refer to a resilient needle guard located in the needle guard housing or to a wall formed with the needle guard housing.

Both the catheter hub 312 and the needle guard housing 300 are made from materials that allow for some compression and expansion of the material, such as from a pliable thermoplastic material. A material or materials with such properties allow for the controlled disengagement of the detent elements 320, 322. A controlled disengagement is one in which the force required for disengagement can be configured at a certain threshold. If the threshold is not met, the detent elements 320, 322 will not disengage.

Method of making and of using the catheter devices discussed elsewhere herein are understood to be within the scope of the present disclosure.

Although limited embodiments of the catheter devices and their components have been specifically described and illustrated herein, many modifications and variations will be apparent to those skilled in the art. Accordingly, it is to be understood that the catheter devices and their components constructed according to principles of the disclosed device, system, and method may be embodied other than as specifically described herein. The disclosure is also defined in the following claims.

What is claimed is:

1. A catheter device comprising:
a catheter hub comprising a pair of wings, an injection port for introducing fluid into the catheter hub and a first detent element;
a needle guard housing in contact with the catheter hub and comprising a second detent element;
a needle hub in contact with the needle guard housing and comprising a needle, the needle comprising a shaft and a distal tip;
wherein engagement between the first detent element and second detent element prevents accidental separation of the catheter hub and needle guard housing; and
wherein the first detent element comprises a groove and the second detent element comprises a projection, the first detent element and the second detent element are both projections, or the first detent element is a projection and the second detent element is a groove.

2. The device of claim 1, wherein the groove having two sidewalls in a v-shape.

3. The device of claim 1, wherein the groove having two sidewalls and a bottom wall.

4. The device of claim 1, wherein the needle guard comprises multiple sub-components that are assembled together.

5. The device of claim 4, wherein one of the detent elements comprises external threads.

6. The device of claim 1, further comprising a tether that unfolds and stretches out between the needle guard housing and the needle hub.

7. The device of claim 1, wherein the needle comprises a crimp and the needle guard housing is provided with an engaging means to engage the crimp.

8. The device of claim 7, wherein the engaging means comprises a perimeter defining an opening.

9. The device of claim 1, wherein the needle guard housing has an open distal end sized and shaped to accept the proximal end of the catheter hub, including a Luer lock.

10. The device of claim 1, wherein the first detent element and the second detent element are both projections and the second detent element is a leaf spring style projection.

11. The device of claim 3, wherein the sidewalls are angled or straight.

12. A method of manufacturing a catheter device, comprising:
forming a catheter hub with a catheter tube; said catheter hub comprising an exterior surface with exterior threads;
forming a needle guard housing with a wall surface having an exterior surface and an interior surface defining an interior cavity;
placing the needle guard housing over a proximal end of the catheter hub such that the proximal end of the catheter projects into the interior cavity of the needle guard housing;
forming a needle hub with a needle having a needle tip and placing the needle hub into engagement with the needle guard housing such that the needle projects through the needle guard housing, the catheter hub, and the catheter tube and the needle guard housing is located inside a shroud formed with the needle hub; and
removably securing the needle guard housing with the catheter hub by engaging a first detent element on the exterior surface of the catheter hub with a second detent element in the interior cavity of the needle guard housing.

13. The method of claim 12, wherein the first detent element is a groove and the second detent element is a projection.

14. The method of claim 12, wherein the first detent element is the exterior threads on the catheter hub.

15. The method of claim 12, further comprising a tether attached to the needle guard housing and the needle hub.

16. The method of claim 12, wherein the needle comprises a change in profile formed proximally of the needle tip for engaging a perimeter defining an opening formed with the needle guard housing.

17. The method of claim 12, wherein the second detent element comprises two leaf springs formed on two sidewalls of the needle guard housing.
